SEQUENCE LISTING (11 sequences)

SEQ ID NO: 1 - RNA, 24 nt, Artificial Sequence
  synthesized RNA
uauaugcuguguguacucugcuuc

SEQ ID NO: 2 - RNA, 24 nt, Artificial Sequence
  synthesized RNA
agcagaguacacacagcauauacc

SEQ ID NO: 3 - RNA, 23 nt, Artificial Sequence
  synthesized RNA
ucaucaucgucucaaaugagucu

SEQ ID NO: 4 - RNA, 36 nt, Artificial Sequence
  synthesized RNA
acuccauuuguuuugaugauggauucuuaugcucca

SEQ ID NO: 5 - RNA, 59 nt, Artificial Sequence
  synthesized RNA
acuccauuuguuuugaugauggauucuuaugcuccaucaucaucgucucaaaugagucu

SEQ ID NO: 6 - RNA, 23 nt, Artificial Sequence
  synthesized RNA
uauaugcuguguguacucugcuu

SEQ ID NO: 7 - RNA, 29 nt, Artificial Sequence
  synthesized RNA
gcagaguacacacagcauauacccaccgg

SEQ ID NO: 8 - RNA, 52 nt, Artificial Sequence
  synthesized RNA
gcagaguacacacagcauauacccaccgguauaugcuguguguacucugcuu

SEQ ID NO: 9 - protein, 334 aa, bacteriophage T4
MFKKYSSLENHYNSKFIEKLYSLGLTGGEWVAREKIHGTNFSLIIERDKVTCAKRTGPILPAEDFFGYEIILKNYADSIKAVQDIMETSAVVSYQVFGEFAGPGIQKNVDYCDKDFYVFDIIVTTESGDVTYVDDYMMESFCNTFKFKMAPLLGRGKFEELIKLPNDLDSVVQDYNFTVDHAGLVDANKCVWNAEAKGEVFTAEGYVLKPCYPSWLRNGNRVAIKCKNSKFSEKKKSDKPIKAKVELSEADNKLVGILACYVTLNRVNNVISKIGEIGPKDFGKVMGLTVQDILEETSREGITLTQADNPSLIKKELVKMVQDVLRPAWIELVS

SEQ ID NO: 10 - protein, 335 aa, Vibrio phage KVP40
MSFVKYTSLENSYRQAFVDKCDMLGVREWVALEKIHGANFSFIVEFKPNEAQDGAEFTVTPAKRTSTIGANVMGDYDFYGCTSVVEAHTAKMEAISNWLWARGIINVGETIIVYGELAGKGVQKEVNYGDKDFWAYDILLPETGKFLDWDVVLEACEFAKVKTTHEIARGTLDELLRIDPLFRSFHTPADVDGDNVAEGFVVKQLRNEKRLHNGSRAILKVKNDKFKEKKNKAGKTPRAAVVLTEEQEKLHAAFSCYLTENRLRNVLSKLETVTQKQFGMISGLFIKDAKDEFERDELNETAIARDDWDVIKRSLTNIANEILRKNWLDILDGNF

SEQ ID NO: 11 - protein, 342 aa, Deinococcus radiorurans
MAERQVIKERAQLFPHPNAERLELCKVGTFQLVVRKGEYRDGDPIVIAPEKAVLPPQLAGLYTNADTGASYLHGAEKNRVGSVRLRGEVSQGVILPLDGLEDAPFGEDLAERLGITFWEPPVPVSMAGEVEPRPPAQHYKHHDVEQFGIYVSEFASGEEVMVTEKLHGTQGVYFRTAEGRWLVTSKGLSRGGLTLREAASNVYWQAARNSNLFAEADAAFSGGEVQIFGEVVPVQKGFSYGQHKPTVFVFKVVYDGLRLPRRDWPQWVLDHAVPVLYEGPFDEATVRKLRGGLETVSGKGLHIREGVVVAPKVPRFAADGSDLSVKLISDAYAKKETGEEYS

(19) United States
(12) Patent Application Publication
(10) Pub. No.: US 2022/0325309 A1
(43) Pub. Date: Oct. 13, 2022

(54) METHOD FOR PRODUCING NUCLEIC ACID MOLECULE

(71) Applicant: SUMITOMO CHEMICAL COMPANY, LIMITED, Chuo-ku (JP)

(72) Inventor: Akihiro SAKATA, Osaka-shi (JP)

(73) Assignee: SUMITOMO CHEMICAL COMPANY, LIMITED, Chuo-ku (JP)

(21) Appl. No.: 17/633,478

(22) PCT Filed: Aug. 8, 2019
(86) PCT No.: PCT/JP2019/031421

§ 371 Date: Feb. 7, 2022

(51) Int. Cl. C12P 19/34

(52) U.S. Cl. CPC C12P 19/34; C12Y 605/01003

(57) ABSTRACT

The present invention provides a method for producing a single-stranded RNA, the method comprising:

(I) a step of reacting a first single-stranded RNA having a phosphate group at the 5′-terminal and a second single-stranded RNA having a hydroxy group at the 3′-terminal with an RNA ligase classified into EC 6.5.1.3 determined by International Union of Biochemistry as an Enzyme Commission number and having a nick repair activity in a double-strand to link said first single-stranded RNA to said second single-stranded RNA; and
(II) a step of purifying the reaction product comprising the single-stranded RNA produced in the step (I) by reverse-phase column chromatography using a mobile phase comprising at least one ammonium salt(s) selected from the group consisting of monoalkylammonium salts and dialkylammonium salts.

### TECHNICAL FIELD

The present invention relates to methods for producing nucleic acid molecules.

### BACKGROUND ART

Patent Documents 1 and 2 disclose single-stranded RNAs to be used in RNA interference methods. Methods for producing such compounds by multistep chemical reactions using nucleic acid synthetic apparatuses are known.

### CITATION LIST

### Patent Document

Patent Document 1: WO 2012/017919 pamphlet
Patent Document 2: WO 2013/103146 pamphlet

### SUMMARY OF THE INVENTION

### Problems to be Solved by Invention

An object of the present invention is to provide simple methods for producing single-stranded RNAs.

### Means to Solve Problems

The present invention includes the following aspects.

A method for producing a single-stranded RNA in one aspect of the present invention is a method for producing a single-stranded RNA, the method comprising

(I) a step of reacting a first single-stranded RNA having a phosphate group at the 5′-terminal and a second single-stranded RNA having a hydroxy group at the 3′-terminal with an RNA ligase classified into EC 6.5.1.3 determined by International Union of Biochemistry as an Enzyme Commission number and having a nick repair activity in a double-strand to link said first single-stranded RNA to said second single-stranded RNA; and
(II) a step of purifying the reaction product comprising the single-stranded RNA produced in the step (I) by reverse-phase column chromatography using a mobile phase comprising at least one ammonium salt(s) selected from the group consisting of monoalkylammonium salts and dialkylammonium salts, wherein

a) said first single-stranded RNA is a single-stranded RNA consisting of a X1 region and a Z region in the order from the 5′-terminal;

b) said second single-stranded RNA is a single-stranded RNA consisting of a X2 region, a Y2 region, a Ly linker region, and a Y1 region in the order from the 5′-terminal;

c) said X1 region and said X2 region are nucleotide sequences consisting of 5 or more nucleotides which are complementary with each other;

d) said Y1 region and said Y2 region are nucleotide sequences consisting of 2 or more nucleotides which are complementary with each other;

e) said Z region is a region comprising a nucleotide sequence having any number of nucleotides;

f) said Ly linker region is a linker region having a 4- to 30-mer nucleotide sequence or an atomic group derivatized from an amino acid; and

g) the single-stranded RNA produced by linking said first single-stranded RNA to said second single-stranded RNA is a ligated single-stranded RNA consisting of said X2 region, said Y2 region, said Ly linker region, said Y1 region, said X1 region, and said Z region in the order from the 5′-terminal.

Also, a method for producing a single-stranded RNA in one aspect of the present invention is said method, wherein

said Z region is a region consisting of a Z1 region, a Lz linker region, and a Z2 region in the order from the 5′-terminal;

said Lz linker region is a linker region having an atomic group derivatized from an amino acid;

said Z1 region and said Z2 region comprise nucleotide sequences which are complementary with each other; and

the single-stranded RNA produced by linking said first single-stranded RNA to said second single-stranded RNA is a ligated single-stranded RNA consisting of said X2 region, said Y2 region, said Ly linker region, said Y1 region, said X1 region, said Z1 region, said Lz linker region, and said Z2 region in the order from the 5′-terminal.

Also, a method for producing a single-stranded RNA in one aspect of the present invention is said method, wherein said Ly linker region is a divalent group represented by the following formula (I).

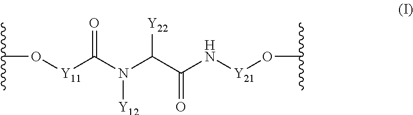

(wherein:

Y₁₁ and Y₂₁ each independently represent an alkylene group having 1 to 20 carbon(s);

Y₁₂ and Y₂₂ each independently represent a hydrogen atom or an alkyl group optionally substituted with an amino group; or

Y₁₂ and Y₂₂ are combined with each other at their terminals to represent an alkylene group having 3 to 4 carbons;

the terminal oxygen atom bound to Y₁₁ is bound to the phosphorus atom of the phosphate ester at the terminal nucleotide in any one region of said Y1 region and said Y2 region; and

the terminal oxygen atom bound to Y₂₁ is bound to the phosphorus atom of the phosphate ester at the terminal nucleotide in the other region of said Y1 region and said Y2 region which is not bound to Y₁₁.)

Also, a method for producing a single-stranded RNA in one aspect of the present invention is said method, wherein

said Ly linker region is a divalent group represented by the following formula (I); and

said Lz linker region is a divalent group represented by the following formula (I′).

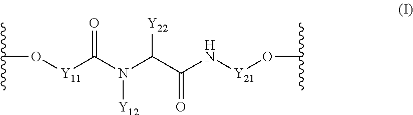

(wherein:

Y₁₁ and Y₂₁ each independently represent an alkylene group having 1 to 20 carbon(s);

Y₁₂ and Y₂₂ each independently represent a hydrogen atom or an alkyl group optionally substituted with an amino group; or

Y₁₂ ? and Y₂₂ are combined with each other at their terminals to represent an alkylene group having 3 to 4 carbons;

the terminal oxygen atom bound to Y₁₁ is bound to the phosphorus atom of the phosphate ester at the terminal nucleotide in any one region of said Y1 region and said Y2 region; and the terminal oxygen atom bound to Y₂₁ is bound to the phosphorus atom of the phosphate ester at the terminal nucleotide in the other region of said Y2 region and said Y1 region which is not bound to Y₁₁.)

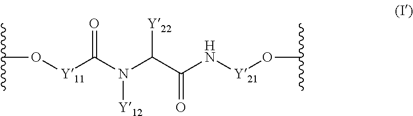

(wherein:

Y′₁₁ and Y′₂₁ each independently represent an alkylene group having 1 to 20 carbon(s);

Y′₁₂ and Y′₂₂ each independently represent a hydrogen atom or an alkyl group optionally substituted with an amino group; or

Y′₁₂ and Y′₂₂ are combined with each other at their terminals to represent an alkylene group having 3 to 4 carbons;

the terminal oxygen atom bound to Y′₁₁ is bound to the phosphorus atom of the phosphate ester at the terminal nucleotide in any one region of said Z1 region and said Z2 region; and

the terminal oxygen atom bound to Y′₂₁ is bound to the phosphorus atom of the phosphate ester at the terminal nucleotide in the other region of said Z2 region and said Z1 region which is not bound to Y′₁₁.)

Also, a method for producing a single-stranded RNA in one aspect of the present invention is said method, wherein said Ly linker region and said Lz linker region each independently represent a divalent group having the structure represented by the following formula (II-A) or (II-B).

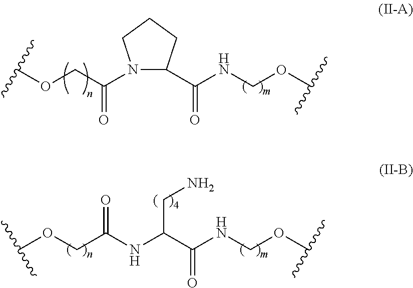

(wherein, n and m each independently represent any one integer of 1 to 20.)

Also, a method for producing a single-stranded RNA in one aspect of the present invention is said method, wherein at least one of a W1 region consisting of said X1 region, said Y1 region, and said Z region, and a W2 region consisting of said X2 region and said Y2 region comprises a nucleotide sequence which suppresses the expression of a target gene in an RNA interference method.

Also, a method for producing a single-stranded RNA in one aspect of the present invention is said method, wherein said RNA ligase is T4 RNA ligase 2 derived from T4 bacteriophage, ligase 2 derived from KVP40, Trypanosoma brucei RNA ligase, Deinococcus radiodurans RNA ligase, or Leishmania tarentolae RNA ligase.

Also, a method for producing a single-stranded RNA in one aspect of the present invention is said method, wherein said RNA ligase is an RNA ligase consisting of an amino acid sequence having 95% or more identity to an amino acid sequence of SEQ ID NO:9, 10, or 11.

Also, a method for producing a single-stranded RNA in one aspect of the present invention is said method, wherein said RNA ligase is T4 RNA ligase 2 derived from T4 bacteriophage or RNA ligase 2 derived from KVP40.

### Effect of Invention

According to the production method of the present invention, single-stranded RNAs can be easily produced.

### BRIEF DESCRIPTION OF DRAWINGS

FIG. 1 is a schematic diagram showing an example of the binding order of each region of a first and a second single-stranded RNAs.

FIG. 2 is a schematic diagram showing an example of a step for producing a ligated single-stranded RNA.

FIG. 3 is a schematic diagram showing an example of the binding order of each region of a first and a second single-stranded RNAs.

FIG. 4 is a schematic diagram showing an example of a step for producing a ligated single-stranded RNA.

FIG. 5 is a chromatogram of an ultraviolet detection method (wavelength: 260 nm) measured by reverse-phase column chromatography using an aqueous solution of triethylammonium acetate as a mobile phase A.

FIG. 6 is a chromatogram of an ultraviolet detection method (wavelength: 260 nm) measured by reverse-phase column chromatography using an aqueous solution of hexylammonium acetate as a mobile phase A in Example 1.

FIG. 7 is a chromatogram of an ultraviolet detection method (wavelength: 260 nm) measured by reverse-phase column chromatography using an aqueous solution of dipropylammonium acetate as a mobile phase A in Example 1.

FIG. 8 is a chromatogram of an ultraviolet detection method (wavelength: 260 nm) measured by reverse-phase column chromatography using an aqueous solution of dibutylammonium acetate as a mobile phase A in Example 1.

FIG. 9 is a chromatogram of an ultraviolet detection method (wavelength: 260 nm) measured by reverse-phase column chromatography using an aqueous solution of diamylammonium acetate as a mobile phase A in Example 1.

FIG. 10 is a chromatogram of an ultraviolet detection method (wavelength: 260 nm) measured by reverse-phase column chromatography using an aqueous solution of tetrabutylammonium phosphate as a mobile phase A in Example 1.

FIG. 11 shows sequences of first and second RNAs used in Examples 1 to 3 and Comparative Examples 1 to 3, and ligated single-stranded RNAs to be produced.

### MODE FOR CARRYING OUT THE INVENTION

A production method in one embodiment of the present invention comprises a step of reacting a first single-stranded RNA having a phosphate group at the 5′-terminal and a second single-stranded RNA having a hydroxy group at the 3′-terminal with an RNA ligase classified into EC 6.5.1.3 determined by International Union of Biochemistry as an Enzyme Commission number and having a nick repair activity in a double-strand to link said first single-stranded RNA and said second single-stranded RNA, and produce a single-stranded RNA. The ligated single-stranded RNA produced in the production method of present embodiment is a single-stranded RNA consisting of a X2 region, a Y2 region, a Ly linker region, a Y1 region, a X1 region, and a Z region from the 5′-terminal. Said Z region may consist of a Z1 region, a Lz linker region, and a Z2 region from the 5′-terminal, and said ligated single-stranded RNA may be a single-stranded RNA consisting of a X2 region, a Y2 region, a Ly linker region, a Y1 region, a X1 region, a Z1 region, a Lz linker region, and a Z2 region. Said ligated single-stranded RNA may comprise a sequence which suppresses the expression of a target gene in an RNA interference method in at least one of a W1 region consisting of a Y1 region, a X1 region, and a Z region (or a Z1 region), and a W2 region consisting of a X2 region and a Y2 region.

RNA interference (RNAi) refers to a phenomenon wherein a double-stranded RNA consisting of a sense RNA consisting of an identical sequence to at least a part of the mRNA sequence of a target gene and an antisense RNA consisting of a complementary sequence thereof is introduced into cells, thereby mRNA of the target gene is degraded, and as a result, inhibition of the translation into a protein is induced, and the expression of the target gene is inhibited. The detailed mechanism of the RNA interference is partially unknown yet, but the main mechanism is believed to include that an enzyme called DICER (one of the RNase III nuclease family) is contacted a double-stranded RNA, and the double-stranded RNA is degraded into small fragments called siRNA.

The target gene is not specifically limited, and a desired gene may be appropriately selected. The nucleotide sequence which suppresses the expression of a target gene is not specifically limited as long as it is a sequence which can suppress the gene expression, and may be designed by a conventional method on the basis of the sequence information of a target gene registered in known database (for example, GenBank) or the like. Said nucleotide sequence has 80% or 85% or more, preferably 90% or more, more preferably 95% or more, still more preferably 98% or more, and the most preferably 100% identity to a prescribed region of a target gene.

As a nucleotide sequence which suppresses the expression of a target gene by RNA interference, for example, a sense RNA consisting of an identical sequence to at least a part of the mRNA sequence of a target gene may be used. The number of bases in the nucleotide sequence which suppresses the expression of a target gene by RNA interference is not specifically limited, and examples thereof include 19 to 30 bases, and preferably 19 to 21 bases.

Any one or both of the W1 region and W2 region may have 2 or more same sequences for suppressing the expression of the same target gene, 2 or more different sequences for suppressing the expression of the same target, or 2 or more different sequences for suppressing the expression of different target genes. When the W1 region has 2 or more sequences for suppressing the expression, the position of each sequence for suppressing the expression is not specifically limited, and may be in any one or both of the X1 region and the Y1 region, or a region over both regions. When the W2 region has 2 or more sequences for suppressing the expression, the position of each sequence for suppressing the expression may be in any one or both of the X2 region and the Y2 region, or a region over both regions. A sequence for suppressing the expression usually has 80% or 85% or more complementation, preferably has 90% or more complementation, more preferably 95%, still more preferably 98%, and especially preferably 100% complementation to a prescribed region of a target gene.

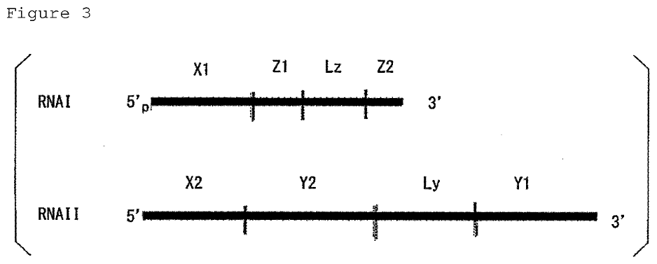

Such ligated single-stranded RNA may be produced by a step of reacting a first single-stranded RNA having a phosphate group at the 5′-terminal and a second single-stranded RNA having a hydroxy group at the 3′-terminal with a ligase, and linking said first single-stranded RNA to said second single-stranded RNA (see FIGS. 1 to 4).

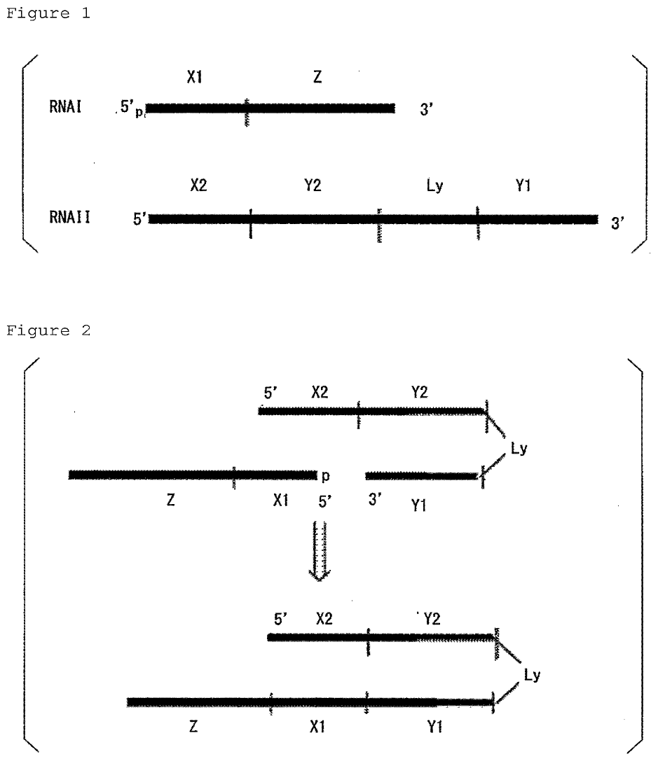

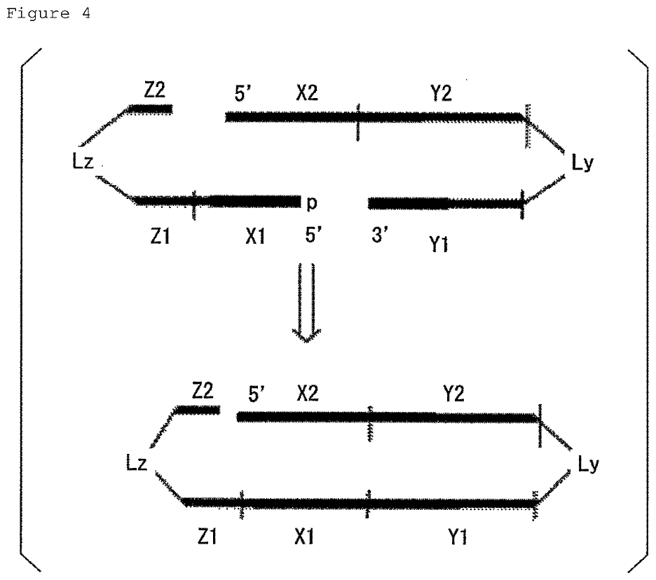

In a ligated single-stranded RNA, complementary sequence moieties may line in the molecule, and may partially form a double-strand in the molecule. As shown in FIG. 2 and FIG. 4, in a ligated single-stranded RNA molecule, the X1 region and the X2 region comprise complementary nucleotide sequences with each other, further the Y1 region and the Y2 region comprise complementary nucleotide sequences with each other, double-strands are formed between these complementary sequences, and the Ly and Lz linker regions form a loop structure depending on the length. FIG. 2 and FIG. 4 just show linking order of said regions and the positional relation of each region forming a double-strand, but, for example, the length of each region, shape of the linker region (Ly and Lz), and the like are not limited to them.

At least one of a W1 region consisting of a Y1 region, a X1 region, and a Z region (or a Z1 region), and a W2 region consisting of a X2 region and a Y2 region may comprise at least one sequence which suppresses the expression of a target gene in an RNA interference method. In a ligated single-stranded RNA, the W1 region and the W2 region may be completely complementary with each other, or noncomplementary in 1 or several nucleotide(s), and preferably completely complementary with each other. Examples of said 1 or several nucleotide(s) include 1 to 7 nucleotide(s), and preferably 1 to 5 nucleotide(s). The Y1 region has a complementary nucleotide sequence to the entire region of the Y2 region. The Y1 region and the Y2 region are completely complementary nucleotide sequences with each other, and nucleotide sequences consisting of a 2 or more same number of nucleotides. The X1 region and the X2 region may be completely complementary with each other, or noncomplementary in 1 to 5 nucleotide(s), and are nucleotide sequences consisting of 2 or more nucleotides. In the production method of present embodiment, the Z region is a region comprising a nucleotide sequence having any number of nucleotide(s) which is not an essential sequence, and the number of nucleotide(s) may be 0 in one aspect, or comprises one or more nucleotide(s) in another aspect. The Z region may be one in which each of the Z1, Lz, and Z2 region is ligated from the 5′-terminal.

Examples of the length of each region include, but are not limited to, the followings. In the present description, the scope of the number of nucleotide(s) discloses, for example, all positive integers which fall within the scope. As a specific example, the term of “1 to 4 nucleotide(s)” refers to the all disclosures of “1 nucleotide”, “2 nucleotides”, “3 nucleotides”, and “4 nucleotides” (the same applies below).

In a ligated single-stranded RNA molecule, the relation between the number of nucleotide(s) in the W2 region (W2n), and the number of nucleotide(s) in the X2 region (X2n) and the number of nucleotide(s) in the Y2 region (Y2n) meets, for example, the requirement of the following equation (1). The relation between the number of nucleotide(s) in the W1 region (W1n), and the number of nucleotide(s) in the X1 region (X1n) and the number of nucleotide(s) in the Y1 region (Y1n) meets, for example, the requirement of the following equation (2).

W2n=X2n+Y2n (1)

W1n≥X1n+Y1n (2)

In a ligated single-stranded RNA molecule, the relation between the number of nucleotide(s) in the X1 region (X1n) and the number of nucleotide(s) in the Y1 region (Y1n) is not specifically limited, and, for example, meets any one requirement of the following formula.

X1n=Y1n (3)

X1n<Y1n (4)

X1n>Y1n (5)

In the method of present embodiment, the number of nucleotide(s) in the X1 region (X1n) and the number of nucleotide(s) in the X2 region (X2n) are 2 or more, preferably 4 or more, and more preferably 10 or more.

The number of nucleotide(s) in the Y1 region (Y1n) and the number of nucleotide(s) in the Y2 region (Y₂n) are 2 or more, preferably 3 or more, and more preferably 4 or more.

The Z1 region preferably comprises a complementary nucleotide sequence to the entire region of the Z2 region or a partial region of the Z2 region. The Z1 region and the Z2 region may be noncomplementary in 1 or several nucleotide(s), but preferably completely complementary with each other.

More specifically, the Z2 region preferably consists of a nucleotide sequence having a shorter nucleotide by 1 or more nucleotide(s) than the Z1 region. In this case, the entire nucleotide sequence of the Z2 region is complementary with all nucleotides in any partial region of the Z1 region. The nucleotide sequence from the 5′-terminal to the 3′-terminal of the Z2 region is more preferably a complementary sequence with a nucleotide sequence from the 3′-terminal nucleotide to the 5′-terminal of the Z1 region.

In a ligated single-stranded RNA molecule, the relation between the number of nucleotide(s) in the X1 region (X1n) and the number of nucleotide(s) in the X2 region (X2n), the relation between the number of nucleotide(s) in the Y1 region (Y1n) and the number of nucleotide(s) in the Y2 region (Y₂n), and the relation between the number of nucleotide(s) in the Z1 region (Z1n) and the number of nucleotide(s) in the Z2 region (Z2n) meet the requirements of the following equations (6), (7), and (8), respectively.

X1n≥X2n (6)

Y1n=Y₂n (7)

Z1n≥Z2n (8)

The full length (total number of nucleotides) of a ligated single-stranded RNA is not specifically limited. In a ligated single-stranded RNA, the lower limit of the total number of nucleotides (the number of full-length nucleotides) is typically 38, preferably 42, more preferably 50, still more preferably 51, and especially preferably 52, and the upper limit thereof is typically 300, preferably 200, more preferably 150, still more preferably 100, and especially preferably 80. In a ligated single-stranded RNA, the lower limit of the total number of nucleotides except for the linker region (Ly, Lz) is typically 38, preferably 42, more preferably 50, still more preferably 51, and especially preferably 52, and the upper limit thereof is typically 300, preferably 200, more preferably 150, still more preferably 100, and especially preferably 80.

In a ligated single-stranded RNA, the lengths of the Ly and Lz linker regions are not specifically limited. These linker regions preferably have lengths so that, for example, the X1 region and the X2 region can form a double-strand or the Y1 region and the Y2 region can form a double-strand.

The upper limit of the number of atoms which forms the main chain of the linker region is typically 100, preferably 80, and more preferably 50.

The Ly linker region is, for example, a divalent group represented by the following formula (I), and the Lz linker region is, for example, a divalent group represented by the following formula (I′).

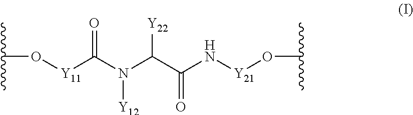

(wherein:

Y₁₁ and Y₂₁ each independently represent an alkylene group having 1 to 20 carbon(s);

Y₁₂ and Y₂₂ each independently represent a hydrogen atom or an alkyl group optionally substituted with an amino group; or

Y₁₂ and Y₂₂ are combined with each other at their terminals to represent an alkylene group having 3 to 4 carbons;

the terminal oxygen atom bound to Y₁₁ is bound to the phosphorus atom of the phosphate ester at the terminal nucleotide in any one region of the Y1 region and the Y2 region; and the terminal oxygen atom bound to Y₂₁ is bound to the phosphorus atom of the phosphate ester at the terminal nucleotide in the other region of the Y1 region and the Y2 region which is not bound to Y₁₁.)

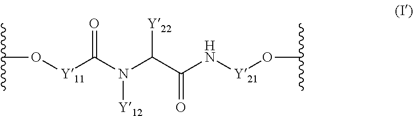

(wherein:

Y′₁₁ and Y′₂₁ each independently represent an alkylene group having 1 to 20 carbon(s);

Y′₁₂ and Y′₂₂ each independently represent a hydrogen atom or an alkyl group optionally substituted with an amino group; or

Y′₁₂ and Y′?₂₂ are combined with each other at their terminals to represent an alkylene group having 3 to 4 carbons;

the terminal oxygen atom bound to Y′₁₁ is bound to the phosphorus atom of the phosphate ester at the terminal nucleotide in any one region of the Z1 region and the Z2 region; and

the terminal oxygen atom bound to Y′₂₁ is bound to the phosphorus atom of the phosphate ester at the terminal nucleotide in the other region of the Z1 region and the Z2 region which is not bound to Y′₁₁.)

The alkylene group having 1 to 20 carbon(s) in said Y₁₁ and Y₂₁, and Y′₁₁ and Y′₂₁ has preferably 1 to 10 carbon(s), and more preferably 1 to 5 carbon(s). The alkylene group may be straight-chain or branched-chain.

The alkyl group optionally substituted with an amino group in said Y₁₂ and Y₂₂, and Y′₁₂ and Y′₂₂ has preferably 1 to 10 carbon(s), and more preferably 1 to 5 carbon(s). The alkyl group may be straight-chain or branched-chain.

Preferable examples of the Ly linker region and the Lz linker region include a divalent group having the structure represented by the following formula (II-A) or (II-B).

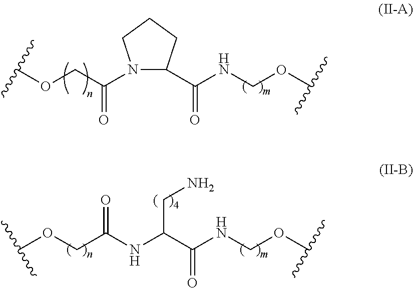

(wherein, n and m each independently represent any one integer of 1 to 20.)

The n and m preferably each independently represent any one integer of 1 to 10, and more preferably any one integer of 1 to 5.

In a preferable aspect, the Ly linker region and the Lz linker region each independently represent any one divalent group of the formula (II-A) or (II-B).

Specific examples of the first single-stranded RNA and the second single-stranded RNA include Chain I and Chain II in FIG. 11.

In the sequences shown in the present description and drawings, the abbreviation of “p” means that the hydroxy group at the 5′-terminal is modified by a phosphate group. Also, the abbreviation of “P” in a sequence is a structure introduced by using an amidite represented by the following structural formula (III-A).

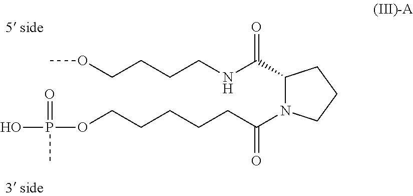

In the method of the present invention, the Ly linker region may be a linker region consisting of a 4- to 30-mer nucleotide sequence. For example, the Ly linker region may be a linker region consisting of a nucleotide sequence having a smaller number of nucleotide(s) than the sum of the number of nucleotides of the X2 region, the Y2 region, the Y1 region, and the X1 region.

More specifically, said Ly linker region may consist of a Lya region, a Lyb region, and a Lyc region from the 5′-terminal, and the Lya region and the Lyc region may be linker regions consisting of nucleotide sequences having 2 bases which do not form a Watson-Crick base pair with each other.

The Ly linker region may consist of a Lya region, a Lyb region, and a Lyc region from the 5′-terminal; the Lyb region may consist of a 0- to 20-mer nucleotide sequence;

the Lya region and the Lyc region may each represent a linker region of a nucleotide sequence having 2 bases; and

the combination of (Lya, Lyc) or (Lyc, Lya) may be selected from the following combinations. (Lya, Lyc) or (Lyc, Lya)=(AA, AA), (AA, AC), (AA, AG), (AA, CA), (AA, CC), (AA, CG), (AA, GA), (AA, GC), (AA, GG), (AC, AA), (AC, AC), (AC, AG), (AC, CA), (AC, CC), (AC, CG), (AC, UA), (AC, UC), (AC, UG), (AG, AA), (AG, AC), (AG, AG), (AG, GA), (AG, GG), (AG, UA), (AG, UC), (AG, UU), (AU, CA), (AU, CC), (AU, CG), (AU, GA), (AU, GC), (AU, GG), (AU, UA), (AU, UC), (AU, UG), (AU, UU), (CA, AA), (CA, AC), (CA, AU), (CC, AA), (CC, AC), (CC, AU), (CC, CC), (CC, CU), (CC, UA), (CC, UC), (CC, UU), (CG, AA), (CG, AC), (CG, AU), (CG, GA), (CG, GC), (CG, GU), (GA, AA), (GA, AG), (GA, GG), (GA, GU), (GC, AA), (GC, AG), (GC, AU), (GC, CA), (GC, CG), (GC, CU), (GC, GA), (GC, GG), (GC, GU), (GC, UA), (GC, UG), (GC, UU), (GG, AA), (GG, AG), (GG, AU), (GG, GA), (GG, GG), (GG, GU), (GG, UU), (GU, CA), (GU, CG), (GU, GU), (GU, UA), (GU, UG), (GU, UU), (UA, AC), (UA, AG), (UA, AU), (UC, AC), (UC, AG), (UC, AU), (UC, CC), (UC, CG), (UC, CU), (UC, UC), (UC, UG), (UC, UU), (UG, AA), (UG, AC), (UG, AG), (UG, AU), (UG, GC), (UG, GG), (UG, GU), (UG, UC), (UG, UG), (UG, UU), (UU, CC), (UU, CG), (UU, CU), (UU, GC), (UU, GG), (UU, GU), (UU, UC), (UU, UG), and (UU, UU)

In the ligation reaction carried out in the production method of present embodiment, an RNA ligase classified into EC 6.5.1.3 determined by International Union of Biochemistry as an Enzyme Commission number and having a nick repair activity in a double-strand (hereinafter also referred to as “Present RNA ligase”) is used. Examples of such RNA ligase include T4 RNA ligase 2 derived from T4 bacteriophage. Said RNA ligase 2 can be, for example, purchased from New England BioLabs. Further, examples of the RNA ligase include ligase 2 derived from vibriophage KVP40, Trypanosoma brucei RNA ligase, Deinococcus radiodurans RNA ligase, or Leishmania tarentolae RNA ligase. As said RNA ligase, for example, one obtained by extraction from each organism and purification according to the method described in a nonpatent document (Structure and Mechanism of RNA Ligase, Structure, Vol. 12, PP. 327-339.) may be used.

As a T4 RNA ligase 2 derived from T4 bacteriophage, an RNA ligase which is a protein consisting of an amino acid sequence having 95% or more identity to the amino acid sequence described in SEQ ID NO:9 and having a nick repair activity in a double-strand may also be used. Examples of such RNA ligase 2 include an enzyme having an amino acid sequence described in SEQ ID NO:9, as well as the mutant T39A, F65A, or F66A (see RNA ligase structures reveal the basis for RNA specificity and conformational changes that drive ligation forward, Cell. Vol. 127, pp. 71-84.). Such RNA ligase 2 may be, for example, obtained according to a method in which Escherichia coli bacteriophage T4 deposited to the ATCC (American Type Culture Collection) as ATCC (registered trademark) 11303 is used on the basis of the disclosures of said document, or a method such as PCR.

The RNA ligase 2 derived from KVP40 may be obtained according to a method described in a nonpatent document (Characterization of bacteriophage KVP40 and T4 RNA ligase 2, Virology, vol. 319, PP. 141-151.). Specifically, for example, said ligase may be obtained according to the following method. Namely, among DNA extracted from bacteriophage KVP40 (for example, deposited to JGI as deposit number Go008199), open reading frame 293 is subjected to digestion with restriction enzymes with NdeI and BamHI, and then the resulting product is amplified by a polymerase chain reaction. The resulting DNA is integrated into a plasmid vector pET16b (Novagen). Alternatively, said DNA sequence may be artificially synthesized by PCR. In this regard, a desired mutant may be obtained by a DNA sequence analysis. Subsequently, a reaction was carried out the resulting vector DNA is integrated into E. coli BL21 (DE3), and cultured in a LB medium comprising 0.1 mg/mL ampicillin. Isopropyl-β-thiogalactoside is added thereto so that the concentration thereof would be 0.5 mM, and the resulting mixture was cultured at 37° C. for 3 hours. All the subsequent procedures are preferably carried out at 4° C. First, fungus bodies are precipitated by a centrifugation procedure, and the resulting precipitates are stored at −80° C. To the frozen fungus bodies is added buffer A [50 mM Tris-HCl (pH 7.5), 0.2 M NaCl, 10° sucrose]. Then, lysozyme and Triton X-100 are added thereto, and the fungus bodies are disrupted by ultrasound so that the target compound would be eluted. Subsequently, a reaction was carried out the target compound is isolated by using affinity chromatography, size exclusion chromatography, or the like. Then, the resulting aqueous solution is subjected to centrifugal filtration, and the eluate is replaced with a buffer so that the solution may be used as a ligase.

An RNA ligase 2 derived from KVP40 may be obtained in this way. As an RNA ligase 2 derived from KVP40, an RNA ligase which is a protein consisting of an amino acid sequence having 95% or more identity to the amino acid sequence of SEQ ID NO:10 and having a nick repair activity in a double-strand may be used.

A Deinococcus radiodurans RNA ligase may be obtained according to the method described in a nonpatent document (An RNA Ligase from Deinococcus radiodurans, J Biol Chem., Vol. 279, No. 49, PP. 50654-61.). For example, said ligase may also be obtained from a biological material deposited to ATCC as ATCC(registered trademark) BAA-816. As a Deinococcus radiodurans RNA ligase, an RNA ligase which is a protein consisting of an amino acid sequence having 95% or more identity to the amino acid sequence of SEQ ID NO:11 and having a nick repair activity in a double-strand may be used. Specific examples of such ligase include an RNA ligase consisting of an amino acid sequence of SEQ ID NO:11, as well as an RNA ligase consisting of an amino acid sequence of the RNA ligase of SEQ ID NO:11 and having a mutation of K165A or E278A (An RNA Ligase from Deinococcus radiodurans, J Biol Chem., Vol. 279, No. 49, PP. 50654-61.).

A Trypanosoma brucei RNA ligase may be obtained according to a method described in a nonpatent document (Association of Two Novel Proteins TbMP52 and TbMP48 with the Trypanosoma brucei RNA Editing Complex, Vol. 21, No. 2, PP. 380-389.).

A Leishmania tarentolae RNA ligase may be obtained according to a method described in a nonpatent document (The Mitochondrial RNA Ligase from Leishmania tarentolae Can Join RNA Molecules Bridged by a Complementary RNA, Vol. 274, No. 34, PP. 24289-24296).

The reaction condition of the production method of present embodiment using the present RNA ligase is not specifically limited as long as it is a condition under which the present RNA ligase works, and typical examples thereof include a condition under which a Tris-HCl buffer (pH 7.5) comprising a first nucleic acid chain, a second nucleic acid chain, ATP, magnesium chloride, and DTT, and pure water are mixed, to the mixed solution is added the present RNA ligase, and then the resulting mixture is reacted at a temperature at which said ligase works (for example, 37° C.) for a prescribed time (for example, 1 hour).

Alternatively, the production method of present embodiment may be carried out according to a condition described in a nonpatent document (Bacteriophage T4 RNA ligase 2 (gp24-1) exemplifies a family of RNA ligases found in all, Proc. Natl. Acad. Sci, 2002, Vol. 99, No. 20, PP. 12709-12714.).

A step wherein said RNA ligase is reacted, and a reaction product comprising a single-stranded RNA produced by linking said first single-stranded RNA to said second single-stranded RNA is purified by reverse-phase column chromatography using a mobile phase comprising at least one ammonium salt(s) selected from the group consisting of monoalkylammonium salts and dialkylammonium salts is described below.

A crude product produced in a ligation reaction of a first single-stranded RNA and a second single-stranded RNA using the present RNA ligase may be isolated according to, for example, a method for firstly precipitating or extracting an RNA. Specifically, a method therein to a solution after the ligation reaction is added a solvent which poorly dissolves an RNA such as ethanol and isopropyl alcohol to precipitate an RNA, or a method wherein a mixed solution of phenol/chloroform/isoamyl alcohol (for example, phenol/chloroform/isoamyl alcohol=25/24/1) is added to a solution after the ligation reaction to extract an RNA in an aqueous layer may be used.

In the step of purifying a produced single-stranded RNA by reverse-phase column chromatography, a mobile phase comprising at least one ammonium salt(s) selected from the group consisting of monoalkylammonium salts and dialkylammonium salts is used. Examples of such ammonium salts typically include ammonium salts consisting of organic or inorganic acids and monoalkylamine or dialkylamine.

In the reverse-phase column chromatography, the mobile phase (eluent) is a non-hydrophobic mobile phase, and specific examples thereof include those comprising the above ammonium salts. Examples of such mobile phase include C1-C3 alcohols (for example, methanol, ethanol, 2-propanol, or n-propanol), nitriles (for example, acetonitrile) and, in some cases, solvents comprising water. Examples of the acids forming the above ammonium salts include carbonic acid, acetic acid, formic acid, trifluoroacetic acid, and propionic acid. Examples of such mobile phase typically include eluents consisting of monoalkylamine or dialkylamine/acetic acid/water/acetonitrile.

Examples of the concentration of said ammonium salts include a concentration of 1-200 mM, 5-150 mM, or 20-100 mM. Examples of the pH range of the mobile phase include a pH range of 6-8 or 6.5-7.5.

The mobile phase may comprise triethylammonium salt or the like other than the above ammonium salts, but the ratio of the at least one ammonium salt(s) selected from the group consisting of monoalkylammonium salts and dialkylammonium salts is, for example, 30 mol % or more, 40 mol % or more, 50 mol % or more, 60 mol % or more, 70 mol % or more, 80 mol % or more, or 90 mol % or more, relative to the total ammonium salts, or consists of at least one ammonium salt(s) only selected from the group consisting of monoalkylammonium salts and dialkylammonium salts.

Specific examples of the at least one ammonium salt(s) to be selected include at least one ammonium salt(s) selected from the group consisting of hexylammonium salts, dipropylammonium salts, dibutylammonium salts, and diamylammonium salts, and ammonium salt(s) selected from them is/are preferably used.

The filler of said reverse-phase column chromatography is a hydrophobic stationary phase, and examples thereof include silica or polymers on which any one or more of a phenyl group, an alkyl group having 1 to 20 carbon(s), or a cyanopropyl group is/are fixed. Examples of such filler of silica or polymers include those having a particle size of 2 μm or more, or 5 μm or more.

The isolation by reverse-phase column chromatography is carried out by passing a mobile phase comprising the above ammonium salt(s) through a column comprising said filler, then passing a solution, in which a single-stranded RNA ligated by a ligase is dissolved, through said mobile phase, binding said RNA to the inside of the column, and then performing a gradient to sequentially increase the organic solvent concentration in the mobile phase to be passed to isolate and elute a target RNA molecule from impurities contained in said RNA (such as unreacted first and/or second RNA chain).

Examples of the temperature of reverse-phase column chromatography is 20-100° C., 25-80° C., or 30-60° C.

Regarding the fractions obtained by reverse-phase column chromatography, the constitution is analyzed by UV absorption of the wavelength of 260 nm under chromatographic conditions generally used in the separation analysis of nucleic acids, the selected fractions are collected, and a purified target compound is obtained. For example, said procedure may be carried out by using a method described in a nonpatent document (Handbook of Analysis of Oligonucleotides and Related Products, CRC Press).

The first single-stranded RNA may be prepared by, for example, a solid-phase synthesis. More specifically, said RNA may be prepared by using a nucleic acid synthesizer (NTS M-4MX-E (manufactured by NIHON TECHNO SERVICE CO., LTD.)) based on the phosphoramidite method. The phosphoramidite method is a method in which a cycle consisting of 3 steps of deblocking, coupling, and oxidation is repeated until a desired base sequence is obtained. Regarding each reagent, for example, a porous glass may be used as a solid-phase support, a solution of dichloroacetic acid in toluene may be used as a deblocking solution, 5-benzylthio-1H-tetrazol may be used as a coupling agent, an iodine solution may be used as an oxidizing agent, and an acetic anhydride solution and an N-methylimidazole solution may be used as capping solutions. The cleavage from the solid-phase support and deprotection after the solid-phase synthesis may be carried out according to, for example, a method described in WO 2013/027843 pamphlet. Namely, an aqueous solution of ammonia and ethanol may be added to the reaction mixture to subject the base moieties and phosphate groups to the deprotection and cleavage from the solid-phase support, then the solid-phase support may be filtered, and then tetrabutylammonium fluoride may be used to carry out the deprotection of 2′-hydroxy group to prepare an RNA.

The amidite used in such solid-phase synthesis is not specifically limited, and for example, an amidite represented by the following structural formula (III-a), wherein R₁ is protected by a tert-butyldimethylsilyl (TBDMS) group, a bis(2-acetoxy)methyl (ACE) group, a (triisopropylsilyloxy)methyl (TOM) group, a (2-cyanoethoxy)ethyl (CEE) group, a (2-cyanoethoxy)methyl (CEM) group, a p-toluylsulfonylethoxymethyl (TEM) group, a (2-cyanoethoxy)methoxymethyl (EMM) group, or the like, i.e., TBDMS amidite (TBDMS RNA Amidites, trade name, ChemGenes Corporation), ACE amidite, TOM amidite, CEE amidite, CEM amidite, TEM amidite (review of Chakhmakhcheva: Protective Groups in the Chemical synthesis of Oligoribonucleotides, Russian Journal of Bioorganic Chemistry, 2013, Vol. 39, No. 1, pp. 1-21.), EMM amidite (disclosed in WO 2013/027843 pamphlet), or the like may also be used.

Also, regarding the Ly linker region and the Lz linker region, an amidite having a proline backbone represented by the following structural formula (III-b) may be used according to the method described in Example A4 of WO 2012/017919 pamphlet. Further, said regions may be similarly prepared by a nucleic acid synthesizer using an amidite represented by any one of the following structural formulae (III-d), and (III-e) (see Examples A1 to A3 of WO 2013/103146 pamphlet).

In the phosphorylation of the 5′-position of the 5′-terminal, an amidite for the phosphorylation of the 5′-terminal may be used in the solid-phase synthesis. As an amidite for the phosphorylation of the 5′-terminal, a commercially available amidite may be used. Also, in the solid-phase synthesis, an RNA molecule wherein the 5′-position of the 5′-terminal is a hydroxy group or a protected hydroxy group may be synthesized, appropriately deprotected, and then phosphorylated by a commercially available phosphorylating agent to prepare a single-stranded RNA having a phosphate group at the 5′-terminal. As the phosphorylating agent, a commercially available Chemical Phosphorylation Reagent (Glen Research) represented by the following structural formula (III-f) is known (patent document EP0816368).

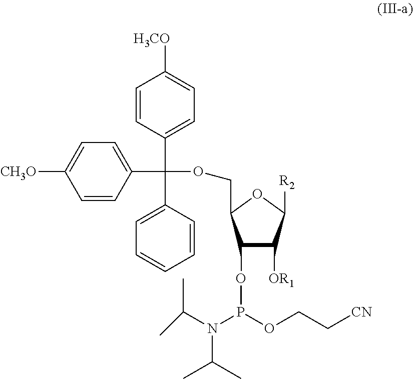

In the formula (III-a), R₂ represents a nucleic acid base optionally protected by a protecting group, and R₁ represents a protecting group.

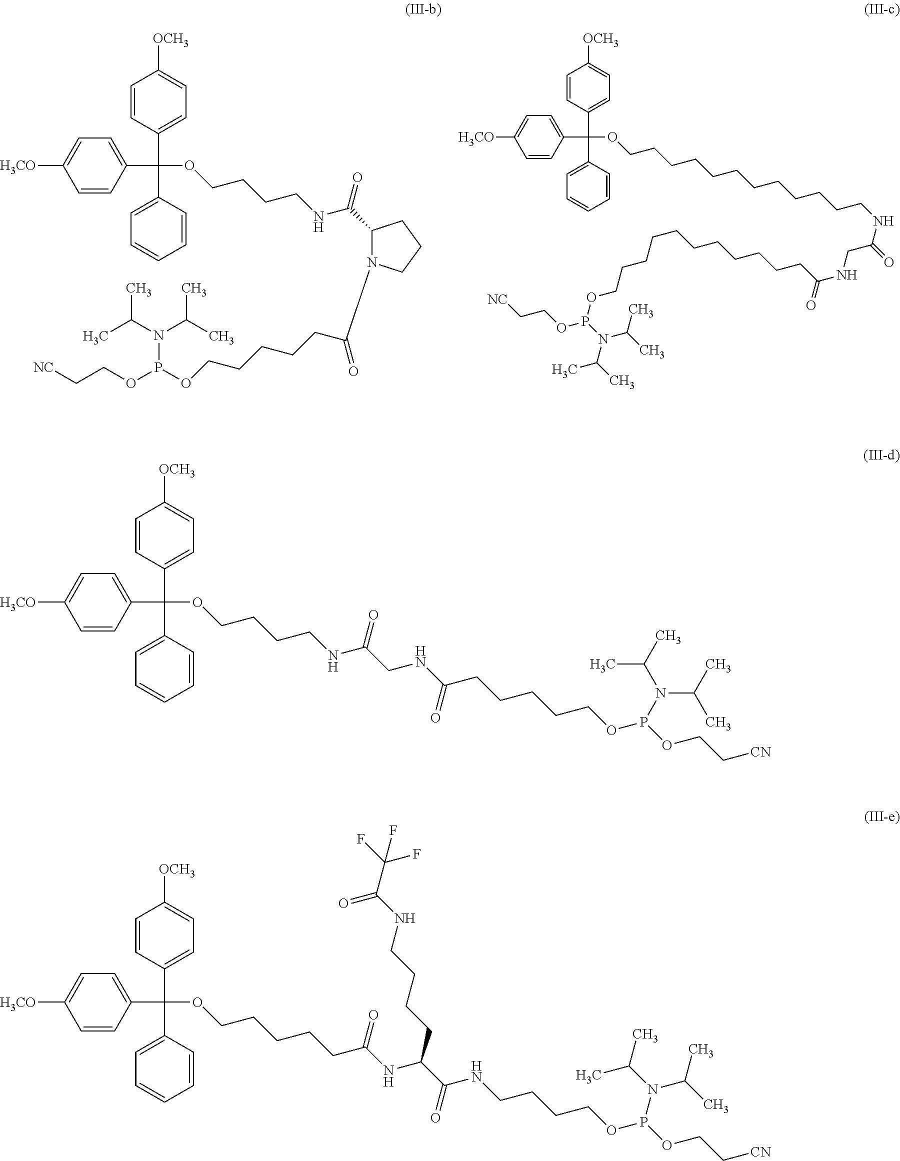 [SMILES: COc1ccc(C(OCCCCNC(=O)[C@@H]2CCCN2C(=O)CCCCCOP(OCCC#N)N(C(C)C)C(C)C)(c2ccccc2)c2ccc(OC)cc2)cc1 ; COc1ccc(C(OCCCCCCCCCCCCNC(=O)CNC(=O)CCCCCCCCCCCOP(OCCC#N)N(C(C)C)C(C)C)(c2ccccc2)c2ccc(OC)cc2)cc1 ; COc1ccc(C(OCCCCNC(=O)CNC(=O)CCCCCOP(OCCC#N)N(C(C)C)C(C)C)(c2ccccc2)c2ccc(OC)cc2)cc1 ; COc1ccc(C(OCCCCCC(=O)N[C@@H](CCCCNC(=O)C(F)(F)F)C(=O)NCCCCOP(OCCC#N)N(C(C)C)C(C)C)(c2ccccc2)c2ccc(OC)cc2)cc1]

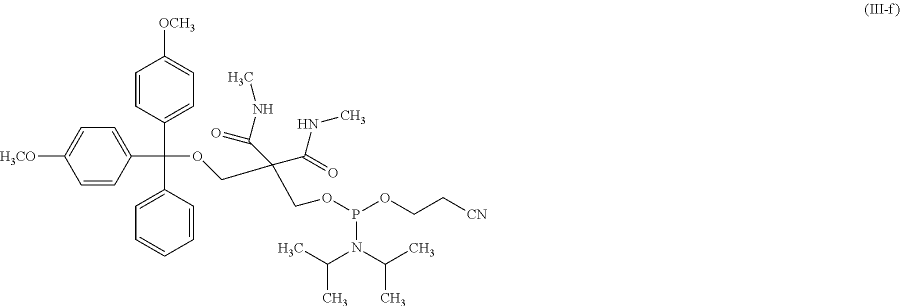 [SMILES: CNC(=O)C(COP(OCCC#N)N(C(C)C)C(C)C)(COC(c1ccccc1)(c1ccc(OC)cc1)c1ccc(OC)cc1)C(=O)NC]

The second single-stranded RNA may be similarly produced by using a solid-phase synthesis, namely using a nucleic acid synthesizer based on the phosphoramidite method.

The bases constituting a nucleotide are usually natural bases constituting a nucleic acid, typically an RNA, but unnatural bases may be used in some cases. Examples of such unnatural bases include modified analogs of natural or unnatural bases.

Examples of the bases include purine bases such as adenine and guanine, and pyrimidine bases such as cytosine, uracil, and thymine. Other examples of the bases include inosine, xanthine, hypoxanthine, nubularine, isoguanisine, and tubercidine. Examples of the above bases include 2-aminoadenine, alkyl derivatives such as 6-methylated purine; alkyl derivatives such as 2-propylated purine; 5-halouracil and 5-halocytosine; 5-propynyluracil and 5-propynylcytosine; 6-azouracil, 6-azocytosine, and 6-azothymine; 5-uracil (pseudouracil), 4-thiouracil, 5-halouracil, 5-(2-aminopropyl)uracil, 5-aminoallyluracil; 8-halogenated, aminated, thiolated, thioalkylated, hydroxylated, and other 8-substituted purines; 5-trifluoromethylated and other 5-substituted pyrimidines; 7-methylguanine; 5-substituted pyrimidine; 6-azapyrimidine; N-2, N-6, and 0-6 substituted purines (including 2-aminopropyladenine); 5-propynyluracil and 5-propynylcytosine; dihydrouracil; 3-deaza-5-azacytosine; 2-aminopurine; 5-alkyluracil; 7-alkylguanine; 5-alkylcytosine; 7-deazaadenine; N6,N6-dimethyladenine; 2,6-diaminopurine; 5-amino-allyl-uracil; N3-methyluracil; substituted 1,2,4-triazole; 2-pyridinone; 5-nitroindole; 3-nitropyrrole; 5-methoxyuracil; uracil-5-oxyacetic acid; 5-methoxycarbonylmethyluracil; 5-methyl-2-thiouracil; 5-methoxycarbonylmethyl-2-thiouracil; 5-methylaminomethyl-2-thiouracil; 3-(3-amino-3-carboxypropyl)uracil; 3-methylcytosine; 5-methylcytosine; N4-acetylcytosine; 2-thiocytosine; N6-methyladenine; N6-isopentyladenine; 2-methylthio-N6-isopentenyladenine; N-methylguanine; and O-alkylated bases. Also, purine bases and pyrimidine bases include, for example, those disclosed in U.S. Pat. No. 3,687,808, “Concise Encyclopedia Of Polymer Science And Engineering”, p. 858-859, edited by Kroschwitz John Wiley & Sons, 1990, and Englisch et al., Angewandte Chemie, International Edition, 1991, vol. 30, p. 613.

The single-stranded RNA nucleic acid molecule consisting of the X2 region, the Y2 region, the Ly linker region, the Y1 region, the X1 region, and the Z region from the 5′-terminal produced in the method of the present embodiment has unligated 5′-terminal and 3′-terminal, i.e., is a linear single-stranded nucleic acid molecule. Said single-stranded RNA nucleic acid molecule may be used, for example for suppressing the expression of a target gene in vivo or in vitro, and may be used for suppressing the expression of a target gene by RNA interference. The term of “suppressing the expression of a target gene” means, for example, the inhibition of the expression of a target gene. The mechanism of said suppression is not specifically limited, and may be, for example, downregulation or silencing.

The suppression of the expression of a target gene can be confirmed by, for example, decrease of the production of transcription product from a target gene, decrease of the activity of transcription product, decrease of the production of translation product from a target gene, decrease of the activity of translation product, or the like. Examples of protein as the translation product include mature proteins and precursor proteins before subjected to processings or post-translational modifications.

### EXAMPLES

Hereinafter, the present invention is illustrated more in detail by way of Examples, but the present invention is not limited to these Examples or the like.

### Example 1

### 1. Synthesis of First Single-Stranded RNA

A single-stranded RNA shown below (Chain I of FIG. 11) was synthesized. Said chain consists of 26 base length, and corresponds to a first single-stranded RNA.

Chain I:
(SEQ ID NO: 1)
pUAUAUGOUGUGUGUACUCUGCUUCPG (5′-3′)

The expression of SEQ ID NO:1 in the SEQUENCE LISTING represents a base sequence from the 5′-terminal before “P”. Said single-stranded RNA was synthesized from the 3′ side to the 5′ side by using a nucleic acid synthesizer (trade name: NTS M-4MX-E, NIHON TECHNO SERVICE CO., LTD.) based on a phosphoramidite method.

In said synthesis, uridine EMM amidite (described in Example 2 of WO 2013/027843 pamphlet), cytidine EMM amidite (described in Example 3 of said document), adenosine EMM amidite (described in Example 4 of said document), guanosine EMM amidite (described in Example 5 of said document), and proline amidite (IIIb) (described in Example A3 of WO 2012/017919 pamphlet) represented by the following formulae respectively were used as RNA amidites, a Chemical Phosphorylation Reagent (Glen Research) represented by the above structural formula (III-f) was used in the 5′ phosphorylation, a porous glass was used as a solid-phase support, a solution of trichloroacetic acid in toluene was used as a deblocking solution, 5-benzylthio-1H-tetrazol was used as a condensing agent, an iodine solution was used as an oxidizing agent, and a phenoxyacetic anhydride solution and a N-methylimidazole solution were used as capping solutions.

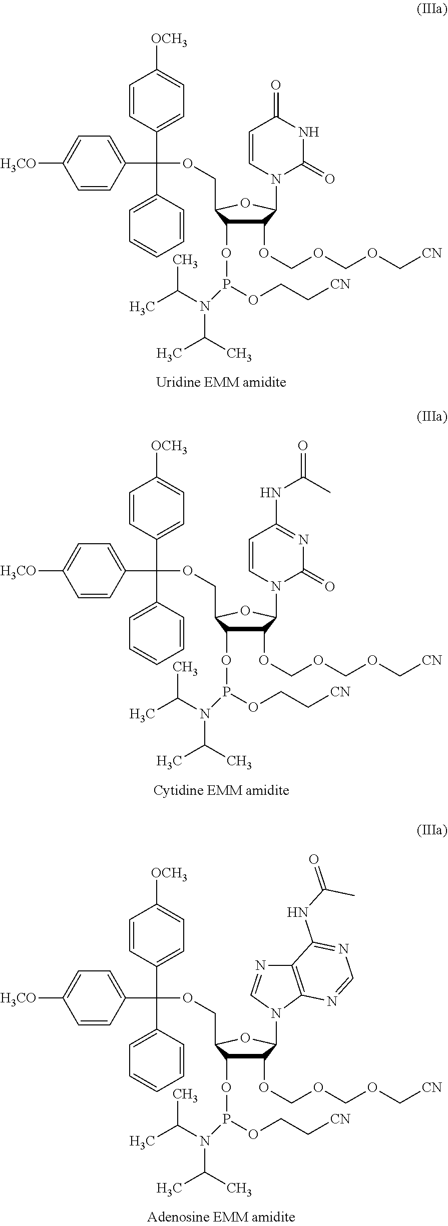

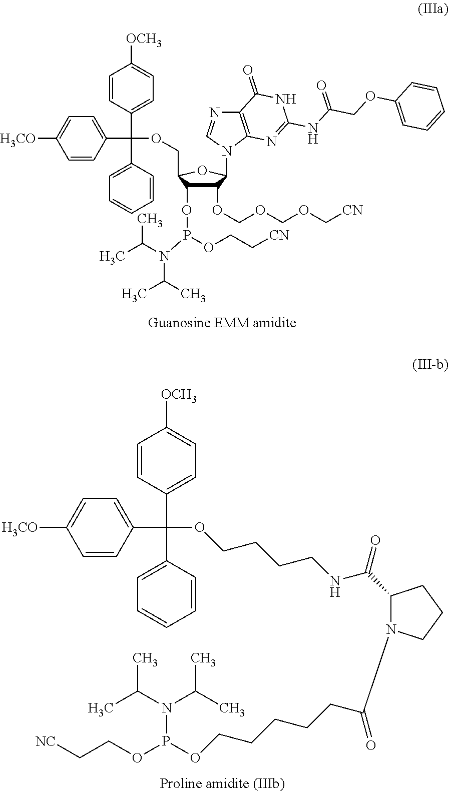

The cleavage from the solid-phase support and deprotection after the solid-phase synthesis was carried out according to a method described in WO 2013/027843 pamphlet. Namely, an aqueous solution of ammonia and ethanol were added to the reaction mixture, the resulting mixture was left to stand for a while, then the solid-phase support was filtered, and the solvent was distilled away. Subsequently, tetrabutylammonium fluoride was used to carry out the deprotection of hydroxy groups. The resulting RNA was dissolved into distilled water for injection so that the concentration thereof would be a desired concentration.

### 2. Synthesis of Second Single-Stranded RNA

A single-stranded RNA shown below (Chain II of FIG. 11) was synthesized. Said chain consists of 27 base length, and corresponds to a second single-stranded RNA.

Chain II:
(SEQ ID NO: 2)
AGCAGAGUACACACAGCAUAUACCPGG (5′-3′)

Said single-stranded RNA was synthesized according to the same method as described above.

The expression of SEQ ID NO:2 in the SEQUENCE LISTING represents a base sequence from the 5′-terminal before “P”. A ligated single-stranded RNA obtained by the ligation of said first and second RNAs is shown in the following Chain III and FIG. 11.

Chain III:
(SEQ ID NO: 1, 2)
AGCAGAGUACACACAGCAUAUACCPGGUAUAUGCUGUGUGUACUCUGCU
UCPG (5′-3′)

In the above sequence, the base sequence from the 5′-terminal base to 27th base corresponds to the sequence of the above SEQ ID NO:2, and the base sequence from the 28th base to the 3′-terminal base corresponds to the sequence of the above SEQ ID NO:1.

### 3. Solid-Phase Synthesis of Single-Stranded RNA Authentic Preparation of Target Compound of Ligation Reaction

A ligated single-stranded RNA authentic preparation obtained by the ligation of the above first and second RNAs was synthesized by a solid-phase synthesis in the same way as said first and second RNAs.

### 4. Investigation of Purification Conditions Using HPLC

The above 3 types of synthesized RNAs were used as authentic preparations, subjected to separation analyses under the conditions shown in Table 1, and the purification conditions were investigated.

TABLE 1

| | |
|---|---|
| Device | Prominence (manufactured |
| | by Shimadzu Corporation) |
| Detection method | UV 260 nm |
| Column | Triart C18 150 mm × 4.6 mm |
| | ID (particle size: 5 μm) |
| | manufactured by YMC |
| Column temperature | 30° C. |
| Flow rate | 1.0 mL/min |

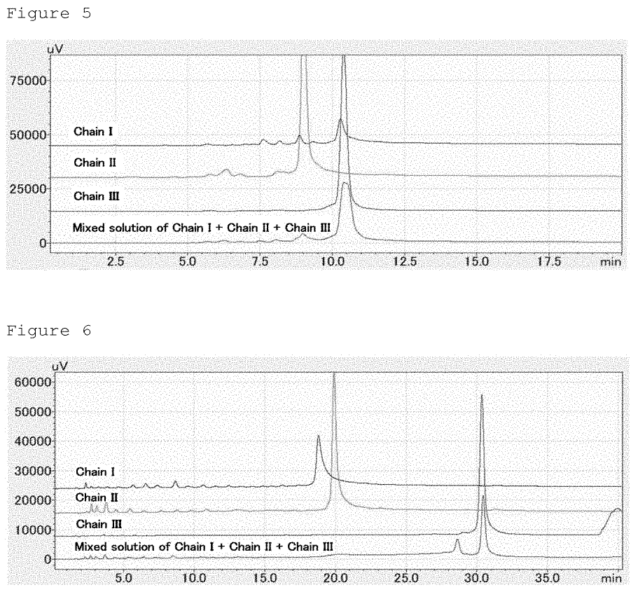

A result of the separation analysis using 100 mM triethylammonium acetate (pH 7.0) as the mobile phase A and acetonitrile as the mobile phase B is shown in FIG. 5. The separation of the starting material Chain I and the product Chain III of the ligation reaction was impossible.

A result of the separation analysis using 100 mM hexylammonium acetate (pH 7.0) as the mobile phase A and acetonitrile as the mobile phase B is shown in FIG. 6. The separation and preparative isolation of the starting materials Chain I and Chain II, and the product Chain III in the ligation reaction were possible.

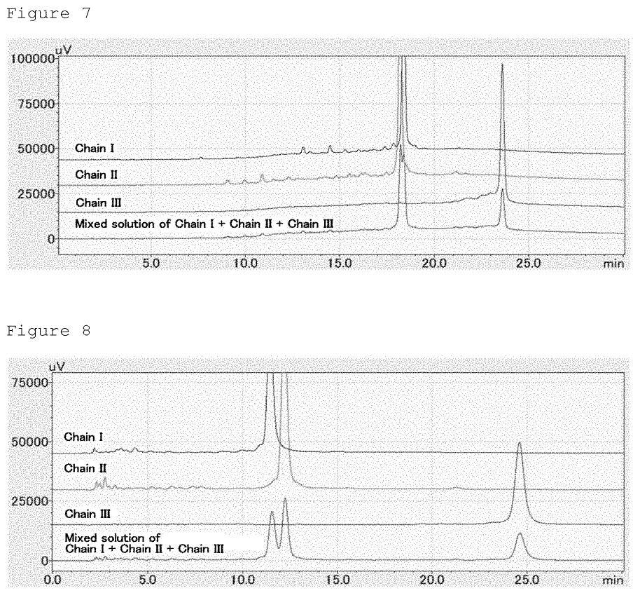

A result of the separation analysis using 100 mM dipropylammonium acetate (pH 7.0) as the mobile phase A and acetonitrile as the mobile phase B is shown in FIG. 7. The separation and preparative isolation of the starting materials Chain I and Chain II, and the product Chain III in the ligation reaction were possible.

A result of the separation analysis using 100 mM dibutylammonium acetate (pH 7.0) as the mobile phase A and acetonitrile as the mobile phase B is shown in FIG. 8. The separation and preparative isolation of the starting materials Chain I and Chain II, and the product Chain III in the ligation reaction were possible.

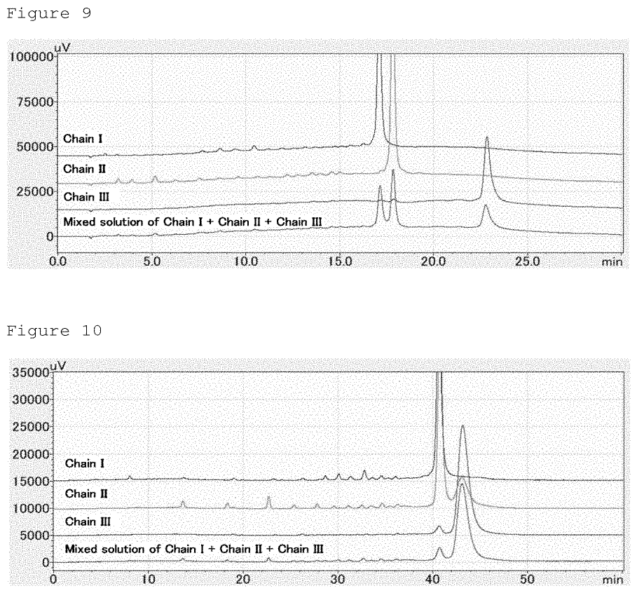

A result of the separation analysis using 100 mM diamylammonium acetate (pH 7.0) as the mobile phase A and acetonitrile as the mobile phase B is shown in FIG. 9. The separation and preparative isolation of the starting materials Chain I and Chain II, and the product Chain III in the ligation reaction were possible.

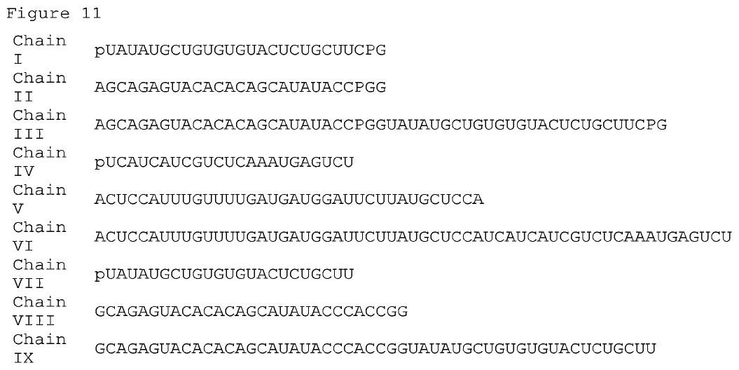

A result of the separation analysis using 10 mM tetrabutylammonium phosphate (pH 7.5) as the mobile phase A and acetonitrile as the mobile phase B is shown in FIG. 10. The separation and preparative isolation of the starting materials Chain I and Chain II, and the product Chain III in the ligation reaction were possible.

### 5. Ligation

Next, to a 50 mL conical tube were added distilled water (manufactured by Otsuka Pharmaceutical Co., Ltd.)

(22.3 mL), 500 mM Tris-Acetate (pH 7.0) (2.8 mL), 0.87 mM Chain I RNA (78.1 μL), and 0.74 mM Chain II RNA (101.3 μL), the resulting mixture was left to stand in a water bath warmed to 65° C. for 10 minutes, and then cooled to room temperature. Subsequently, a reaction was carried out at a constitution of 1625 units T4 RNA ligase 2 (New England Biolabs), 20 mM MgCl₂, 10 mM DTT, and 4 mM ATP mixed solution (2.8 mL), and at a reaction scale of 28.2 mL. Subsequently, the reaction solution was incubated at 37° C. for 1 hour, a 0.2 M aqueous solution of ethylenediaminetetraacetic acid (1 mL) was added to the reaction solution, and the reaction solution was left to stand in a water bath at 65° C. for 10 minutes to stop the reaction.

A part of the resulting reaction solution was taken out, and analyzed by HPLC to confirm that the purity of the target compound in the crude product was 61%, and residual ratios of the Chain I and Chain II were 6.4% and 8.3%, respectively. In the HPLC, each chain was detected by a UV spectrum at a wavelength of 260 nm, the purity was calculated as the area value of the target compound relative to the total area value of the resulting chromatogram, and the residual ratio was calculated as the area value of the starting material relative to the total area value.

Subsequently, said reaction solution (14 mL) was taken out, filtered by using Millex-GP (Merck & Co.), and washed with 100 mM hexylammonium acetate (pH 7.0) (1 mL).

### 6. Purification

Purification by column chromatography was carried out under the conditions described in the following Table 2. The mobile phase A and the mobile phase B were passed through the column at a ratio of 65/35 and at a flow rate of 1.0 mL/min for 10 minutes before purification, and then the sample was added thereto. Each resulting fraction was analyzed by HPLC. In the HPLC, each chain was detected by a UV spectrum at a wavelength of 260 nm, and the purity was calculated as the area value of the target compound relative to the total area value of the resulting chromatogram.

As a result, the fraction comprising the highest purity of the target compound among the resulting fractions showed a purity of 96.4%. As a result of measuring the resulting sample by mass spectrometry measurement, as shown in Table 3, the measured value accorded with the calculated value to confirm that the target compound was obtained. Also, the starting materials Chain I and Chain II were not detected.

TABLE 2

| Device | AKTA purifier 100 PLUS | | | | |
|---|---|---|---|---|---|
| | manufactured by GE Healthcare) | | | | |
| Detection method | UV 260 nm | | | | |
| Column | Triart C18 150 mm × 4.6 mm ID | | | | |
| | (particle size: 5 μm) manufactured by YMC | | | | |
| Separation mode | reverse-phase chromatography | | | | |
| Column temperature | 30° C. | | | | |
| Flow rate | 1.0 mL/min | | | | |
| Mobile phase A | 100 mM hexylammonium acetate aqueous | | | | |
| | solution (pH 7.0) | | | | |
| Mobile phase B | acetonitrile | | | | |
| Preparative isolation width | 1.25 mL | | | | |
| Gradient condition | Time (min) | 0 | 25 | 25.1 | 40 |
| | Mobile phase B | 35 | 55 | 100 | 100 |
| | concentration (%) | | | | |

TABLE 3

| Mass spectrometry result | Chain I | Chain II | Target compound |
|---|---|---|---|
| Theoretical value | 8329.0980 | 8714.3715 | 17025.4589 |
| Comparative Example 1 | 8329.1180 | 8714.3810 | 17025.5040 |
| Example 1 | N.D. | N.D. | 17025.5000 |

N.D.: Not detected.

### Comparative Example 1

14 mL of the reaction solution obtained in the ligation in Example 1 was taken out, filtered by using Millex-GP (manufactured by Merck & Co.), and washed with 100 mM triethylammonium acetate (pH 7.0) (1 mL).

Purification by column chromatography was carried out under the conditions described in the following Table 4. The mobile phase A and the mobile phase B were passed through the column at a ratio of 95/5 and at a flow rate of 1.0 mL/min for 10 minutes before purification, and then the sample was added thereto. The resulting fractions were analyzed by HPLC. The purity was calculated according to the same method as described in Example 1.

As a result, the fraction comprising the highest purity of the target compound showed a purity of 77%. The resulting sample was subjected to mass spectrometry measurement, and the result is shown in Table 3.

TABLE 4

| Device | AKTA purifier 100 | | | | |
|---|---|---|---|---|---|
| | (manufactured by GE Healthcare) | | | | |
| Detector | UV 260 nm | | | | |
| Column | Triart C18 150 mm × 4.6 mm | | | | |
| | ID manufactured by YMC | | | | |
| Separation mode | reverse-phase chromatography | | | | |
| Column temperature | 30° C. | | | | |
| Flow rate | 1.0 mL/min | | | | |
| Mobile phase A | 100 mM triethylammonium | | | | |
| | acetate (pH 7.0) | | | | |
| Mobile phase B | acetonitrile | | | | |
| Preparative isolation width | 1.25 mL | | | | |
| Gradient condition | Time (min) | 0 | 25 | 25.1 | 40 |
| | Mobile phase B | 5 | 25 | 100 | 100 |
| | concentration (%) | | | | |

### Reference Example 1

### 5. Ligation

Next, to a 250 mL polypropylene reactor were added distilled water (manufactured by Otsuka Pharmaceutical Co., Ltd.) (68 mM), 500 mM Tris-Acetate (pH 7.0) (8.6 mL), 0.87 mM Chain I RNA (203 μL), and 0.74 mM Chain II RNA (230 μL), the resulting mixture was left to stand in a water bath warmed to 65° C. for 10 minutes, and then cooled to room temperature. Subsequently, a reaction was carried out at a constitution of 2500 units T4 RNA ligase 2 (New England Biolabs), 20 mM MgCl₂, 10 mM DTT, and 4 mM ATP mixed solution (2.8 mL), and at a reaction scale of 28.2 mL. Subsequently, the reaction solution was incubated at 35° C. for 24 hours, a 0.2 M aqueous solution of ethylenediaminetetraacetic acid (1 mL) was added to the reaction solution, and the reaction solution was left to stand in a water bath at 65° C. for 10 minutes to stop the reaction.

Subsequently, 1 mL of the resulting reaction solution was taken out, filtered by using Millex-GP (Merck & Co.), and washed with distilled water (manufactured by Otsuka Pharmaceutical Co., Ltd.) (1 mL).

### 6. Purification

Purification by column chromatography was carried out under the conditions described in the following Table 5. The mobile phase A and the mobile phase B were passed through the column at a ratio of 65/35 and at a flow rate of 1.0 mL/min for 10 minutes before purification, and then the sample was added thereto. Each resulting fraction was analyzed by HPLC, and the purity and residual ratio were calculated according to the same method as described in Example 1.

As a result, the fraction comprising the highest purity of the target compound among the resulting fractions showed a purity of 79%, and the residual ratios of the Chain I and Chain II were 5.0% and 7.1% respectively.

TABLE 5

| Device | AKTA purifier 100 | | | | |
|---|---|---|---|---|---|
| | (manufactured by GE Healthcare) | | | | |
| Detector | UV 260 nm | | | | |
| Column | Resource 15Q 1 ml manufactured | | | | |
| | by GE Healthcare | | | | |
| Separation mode | anion exchange chromatography | | | | |
| Column temperature | 40° C. | | | | |
| Flow rate | 2.0 mL/min | | | | |
| Mobile phase A | 20 mM sodium phosphate buffer (pH 7.4) | | | | |
| Mobile phase B | 20 mM sodium phosphate + 1M | | | | |
| | sodium chloride aqueous solution (pH 7.4) | | | | |
| Preparative isolation width | 1.0 ml | | | | |
| Gradient condition | Time (min) | 0 | 30 | 30.1 | 45 |
| | Mobile phase B | 0 | 60 | 100 | 100 |
| | concentration (%) | | | | |

These results are summarized in Table 6. Ion exchange chromatography (hereinafter also abbreviated as “ion exchange”) and reverse-phase chromatography using triethylammonium acetate (hereinafter also abbreviated as “TEAR purification”) eluted the target compound and the unreacted starting materials simultaneously, meanwhile reverse-phase chromatography using hexylammonium acetate (hereinafter also abbreviated as “HAA purification”) could separate the target compound and the unreacted starting materials.

TABLE 6

| | | | | Target |
|---|---|---|---|---|
| | | Chain | Chain | compound |
| | | I | II | (Chain III) |
| | Before purification | 6.4% | 8.3% | 61.3% |
| Comparative Example 1 | TEAA purification | 5.0% | 6.2% | 77.3% |
| Example 1 | HAA purification | N.D. | N.D. | 96.4% |
| Reference Example 1 | ion exchange | 5.0% | 7.1% | 78.9% |

### Example 2

### 1. Synthesis of First Single-Stranded RNA

As a first single-stranded RNA, a single-stranded RNA shown below (Chain IV of FIG. 11) was used. Said chain consists of 23 base length, and corresponds to a first single-stranded RNA.

Chain IV: pUCAUCAUCGUCUCAAAUGAGUCU (5′-3′) (SEQ ID NO:3)

Said single-stranded RNA was purchased from Sigma-Aldrich Japan.

### 2. Synthesis of Second Single-Stranded RNA

As a second single-stranded RNA, a single-stranded RNA shown below (Chain V of FIG. 11) was used. Said chain consists of 36 base length, and corresponds to a second single-stranded RNA.

Chain V:
(SEQ ID NO: 4)
ACUCCAUUUGUUUUGAUGAUGGAUUCUUAUGCUCCA (5′-3′)

Said single-stranded RNA was purchased from Sigma-Aldrich Japan.

A ligated single-stranded RNA obtained by the ligation of the above first and second RNAs is shown in the following and FIG. 11.

Chain VI:
(SEQ ID NO: 5)
ACUCCAUUUGUUUUGAUGAUGGAUUCUUAUGCUCCAUCAUCAUCGUCUC
AAAUGAGUCU (5′-3′)

In the above sequence, the base sequence from the 5′-terminal base to the 36th base corresponds to the sequence of the above SEQ ID NO:4, and the base sequence from the 37th base to the 3′-terminal base corresponds to the sequence of the above SEQ ID NO:3.

### 3. Ligation

Next, to a 50 mL conical tube were added distilled water (manufactured by Otsuka Pharmaceutical Co., Ltd.) (1.74 mL), 500 mM Tris-Acetate (pH 7.0) (234 μL), 0.10 mM Chain IV RNA (64.1 μL), and 0.10 mM Chain V RNA (70.5 μL), the resulting mixture was left to stand in a water bath warmed to 65° C. for 10 minutes, and then cooled to room temperature. Subsequently, a reaction was carried out at a constitution of 1750 units T4 RNA ligase 2 (New England Biolabs), 20 mM MgCl₂, 10 mM DTT, and 4 mM ATP mixed solution (234 μL), and at a reaction scale of 2.4 mL. Subsequently, the reaction solution was incubated at 37° C. for 1 hour, a 0.2 M aqueous solution of ethylenediaminetetraacetic acid (0.1 mL) was added to the reaction solution, and the reaction solution was left to stand in a water bath at 65° C. for 10 minutes to stop the reaction.

A part of the resulting reaction solution was taken out, analyzed by HPLC, and the purity and residual ratio were calculated according to the same method as described in Example 1. As a result, the purity of the target compound in the crude product was 29.3%, and the residual ratios of the Chain IV and Chain V were 13.8% and 11.8%, respectively.

Subsequently, 1.2 mL of said reaction solution was taken out, filtered by using Millex-GP (Merck & Co.), and washed with 100 mM hexylammonium acetate (pH 7.0) (1 mL).

### 4. Purification

Purification by column chromatography was carried out under the conditions described in Table 2. The mobile phase A and the mobile phase B were passed through the column at a ratio of 65/35 and at a flow rate of 1.0 mL/min for 10 minutes before purification, and then the sample was added thereto. Each resulting fraction was analyzed by HPLC, and the purity was calculated according to the same method as described in Example 1. As a result, the fraction comprising the highest purity of the target compound among the resulting fractions showed a purity of 80.5% of the target compound. The result of measuring the resulting sample by mass spectrometry measurement is shown in Table 7. The measured value accorded with the calculated value to confirm that the target compound was obtained. Also, the mass spectrometry could not detect the starting materials Chain IV and Chain V.

TABLE 7

| Mass spectrometry result | Chain IV | Chain V | Target compound |
|---|---|---|---|
| Theoretical value | 7305.9180 | 11343.3822 | 18631.2896 |
| Comparative Example 2 | 7305.940 | 11343.448 | 18631.373 |
| Example 2 | N.D. | N.D. | 18631.364 |

### Comparative Example 2

In Example 2, 1.2 mL of the reaction solution obtained by stopping the ligation was taken out, filtered by using Millex-GP (manufactured by Merck & Co.), and washed with 100 mM triethylammonium acetate (pH 7.0) (1 mL).

Purification by column chromatography was carried out under the conditions described in Table 4. The mobile phase A and the mobile phase B were passed through the column at a ratio of 95/5 and at a flow rate of 1.0 mL/min for 10 minutes before purification, and then the sample was added thereto. Each resulting fraction was analyzed by HPLC, and the purity and residual ratio were calculated according to the same method as described in Example 1. As a result, the fraction comprising the highest purity of the target compound showed a purity of 47.1%, and the residual ratios of the Chain IV and Chain V were 1.9% and 13.7%, respectively. As a result of measuring the resulting sample by mass spectrometry measurement, the measured value accorded with the calculated value to confirm that the target compound was obtained. The result of the mass spectrometry measurement of the resulting sample is shown in Table 7.

The purity and the residual ratios of the starting materials in each fraction comprising the highest purity of the target compound in Comparative Example 2 and Example 2 are summarized in Table 8.

TABLE 8

| | | | | Target |
|---|---|---|---|---|
| | | Chain | Chain | compound |
| | | IV | V | (Chain VI) |
| | Before purification | 13.8% | 11.8% | 29.3% |
| Comparative Example 2 | TEAA purification | 1.9% | 13.7% | 47.1% |
| Example 2 | HAA purification | N. D. | 4.0% | 80.5% |

### Example 3

### 1. Synthesis of First Single-Stranded RNA

A single-stranded RNA shown below (Chain VII of FIG. 11) was synthesized. Said chain consists of 23 base length, and corresponds to a first single-stranded RNA.

Chain VII:
(SEQ ID NO: 6)
pUAUAUGCUGUGUGUACUCUGCUU (5′-3′)

Said single-stranded RNA was synthesized from the 3′ side to the 5′ side according to the same method as the synthesis of the single-stranded RNA (Chain I) in Example 1 based on a phosphoramidite method using a nucleic acid synthesizer (trade name: NTS M-4MX-E, NIHON TECHNO SERVICE CO., LTD.), and further the cleavage from the solid-phase support and deprotection after the solid-phase synthesis were carried out according to the same method as described in Example 1.

### 2. Synthesis of Second Single-Stranded RNA

A single-stranded RNA shown below (Chain VIII of FIG. 11) was synthesized according to the same method as the above first single-stranded RNA. Said chain consists of 29 base length, and corresponds to a second single-stranded RNA.

Chain VIII:
(SEQ ID NO: 7)
GCAGAGUACACACAGCAUAUACCCACCGG (5′-3′)

A ligated single-stranded RNA obtained by the ligation of the above first and second RNAs is shown in the following.

Chain IX:
(SEQ ID NO: 8)
GCAGAGUACACACAGCAUAUACCCACCGGUAUAUGCUGUGUGUACUCUGC
UU (5′-3′)

In the above sequence, the base sequence from the 5′-terminal base to the 29th base corresponds to the sequence of the above SEQ ID NO:7, and the base sequence from the 30th base to the 3′-terminal base corresponds to the sequence of the above SEQ ID NO:6.

### 3. Ligation

Next, to a 50 mL conical tube were added distilled water (manufactured by Otsuka Pharmaceutical Co., Ltd.) (19.6 mL), 500 mM Tris-Acetate (pH 7.0) (2.5 mL), 0.62 mM Chain VII RNA (111.2 μL), and 0.49 mM Chain VIII RNA (154.9 μL), the resulting mixture was left to stand in a water bath warmed to 65° C. for 10 minutes, and then cooled to room temperature. Subsequently, a reaction was carried out at a constitution of 1750 units T4 RNA ligase 2 (New England Biolabs), 20 mM MgCl₂, 10 mM DTT, and 4 mM ATP mixed solution (2.5 mL), and at a reaction scale of 25 mL. Subsequently, the reaction solution was incubated at 37° C. for 1 hour, a 0.2 M aqueous solution of ethylenediaminetetraacetic acid (1 mL) was added to the reaction solution, and the reaction solution was left to stand in a water bath at 65° C. for 10 minutes to stop the reaction.

A part of the resulting reaction solution was taken out, and the purity and residual ratio were calculated according to the same method as described in Example 1. As a result, the purity of the target compound in the crude product was 33.0%, and the residual ratios of the Chain VII and Chain VIII were 15.7% and 28.1%, respectively.

Subsequently, 12.5 mL of said reaction solution was taken out, filtered by using Millex-GP (Merck & Co.), and washed with 100 mM hexylammonium acetate (pH 7.0) (1 mL).

### 4. Purification

Purification by column chromatography was carried out under the conditions described in Table 2. The mobile phase A and the mobile phase B were passed through the column at a ratio of 65/35 and at a flow rate of 1.0 mL/min for 10 minutes before purification, and then the sample was added thereto. Each resulting fraction was analyzed by HPLC, and the purity and residual ratio were calculated according to the same method as described in Example 1. As a result, the fraction comprising the highest purity of the target compound showed a purity of 94.2%, and the residual ratios of the Chain VII and Chain VIII were 0.4% and 0.5%, respectively. The result of measuring the resulting sample by mass spectrometry measurement is shown in Table 9. The measured value accorded with the calculated value to confirm that the target compound was obtained.

TABLE 9

| Mass spectrometry result | Chain VII | Chain VIII | Target compound |
|---|---|---|---|
| Theoretical value | 7316.8486 | 9267.3347 | 16566.1727 |
| Comparative Example 3 | 7316.876 | 9267.368 | 16566.282 |
| Example 3 | N.D. | N.D. | 16566.255 |

### Comparative Example 3

12.5 mL of the reaction solution comprising the single-stranded RNA obtained in the ligation reaction in Example 3 was taken out, filtered by using Millex-GP (manufactured by Merck & Co.), and washed with 100 mM triethylammonium acetate(pH 7.0) (1 mL).

Purification by column chromatography was carried out under the conditions described in Table 4. The mobile phase A and the mobile phase B were passed through the column at a ratio of 95/5 and at a flow rate of 1.0 mL/min for 10 minutes before purification, and then the sample was added thereto. Each resulting fraction was analyzed by HPLC, and the purity and residual ratio were calculated according to the same method as described in Example 1. As a result, the fraction comprising the highest purity of the target compound showed a purity of 35.5%, and the residual ratios of the Chain VII and Chain VIII were 17.0% and 30.1%, respectively. As a result of measuring the resulting sample by mass spectrometry measurement, the measured value accorded with the calculated value to confirm that the target compound was obtained. The result of the mass spectrometry measurement of the resulting sample is shown in Table 9.

The purity and the residual ratios of the starting materials in each fraction comprising the highest purity of the target compound in Comparative Example 3 and Example 3 are summarized in Table 10.

TABLE 10

| | | | | Target |
|---|---|---|---|---|
| | | Chain | Chain | compound |
| | | VII | VIII | (Chain IX) |
| | Before purification | 15.7% | 28.1% | 33.3% |
| Comparative Example 3 | TEAA purification | 17.0% | 30.1% | 35.5% |
| Example 3 | HAA purification | 0.4% | 0.5% | 94.2% |

TABLE 11

| | | | Number of nucleotide(s) in | | | | | |
|---|---|---|---|---|---|---|---|---|
| | | Chain | each region | | | | | |
| | RNA chain | length | X1 | X2 | Y1 | Y2 | Z | Ly |
| Ex. 1 | Chain I: SEQ ID | 26 | 22 | — | — | — | 4 | — |
| | NO:1 | | | | | | | |
| | Chain II: SEQ ID | 27 | — | 22 | 2 | 2 | — | P |
| | NO:2 | | | | | | | |
| | Chain III: SEQ ID | 53 | 22 | 22 | 2 | 2 | 4 | P |
| | NO:1,2 | | | | | | | |
| Ex. 2 | Chain IV: SEQ ID | 23 | 21 | — | — | — | 2 | — |
| | NO:3 | | | | | | | |
| | Chain V: SEQ ID | 36 | — | 18 | 7 | 7 | — | 4 |
| | NO:4 | | | | | | | |
| | Chain VI: SEQ ID | 59 | 21 | 18 | 7 | 7 | 2 | 4 |
| | NO:5 | | | | | | | |
| Ex. 3 | Chain VII: SEQ ID | 23 | 21 | — | — | — | 2 | — |
| | NO:6 | | | | | | | |
| | Chain VIII: SEQ | 29 | — | 21 | 2 | 2 | — | 4 |
| | ID NO:7 | | | | | | | |
| | Chain IX: SEQ ID | 52 | 21 | 21 | 2 | 2 | 2 | 4 |
| | NO:8 | | | | | | | |

### INDUSTRIAL APPLICABILITY

According to the production method of the present invention, a single-stranded RNA can be easily produced.

### SEQUENCE LISTING FREE TEXT

SEQ ID NO:1-8 show base sequences of RNA.

SEQ ID NO:9-11 show amino acid sequences.

1. A method for producing a single-stranded RNA, the method comprising:
(I) a step of reacting a first single-stranded RNA having a phosphate group at the 5′-terminal and a second single-stranded RNA having a hydroxy group at the 3′-terminal with an RNA ligase classified into EC 6.5.1.3 determined by International Union of Biochemistry as an Enzyme Commission number and having a nick repair activity in a double-strand to link said first single-stranded RNA to said second single-stranded RNA; and
(II) a step of purifying the reaction product comprising the single-stranded RNA produced in the step (I) by reverse-phase column chromatography using a mobile phase comprising at least one ammonium salt(s) selected from the group consisting of monoalkylammonium salts and dialkylammonium salts, wherein
a) said first single-stranded RNA is a single-stranded RNA consisting of a X1 region and a Z region in the order from the 5′-terminal;
b) said second single-stranded RNA is a single-stranded RNA consisting of a X2 region, a Y2 region, a Ly linker region, and a Y1 region in the order from the 5′-terminal;
c) said X1 region and said X2 region are nucleotide sequences consisting of 5 or more nucleotides which are complementary with each other;
d) said Y1 region and said Y2 region are nucleotide sequences consisting of 2 or more nucleotides which are complementary with each other;
e) said Z region is a region comprising a nucleotide sequence having any number of nucleotides;
f) said Ly linker region is a linker region having a 4- to 30-mer nucleotide sequence or an atomic group derivatized from an amino acid; and
g) the single-stranded RNA produced by linking said first single-stranded RNA to said second single-stranded RNA is a ligated single-stranded RNA consisting of said X2 region, said Y2 region, said Ly linker region, said Y1 region, said X1 region, and said Z region in the order from the 5′-terminal.

2. The method according to claim 1, wherein
said Z region is a region consisting of a Z1 region, a Lz linker region, and a Z2 region in the order from the 5′-terminal;
said Lz linker region is a linker region having an atomic group derivatized from an amino acid;
said Z1 region and said Z2 region comprise nucleotide sequences which are complementary with each other; and
the single-stranded RNA produced by linking said first single-stranded RNA to said second single-stranded RNA is a single-stranded RNA consisting of said X2 region, said Y2 region, said Ly linker region, said Y1 region, said X1 region, said Z1 region, said Lz linker region, and said Z2 region in the order from the 5′-terminal.

3. The method according to claim 1, wherein
said Ly linker region is a divalent group represented by the following formula (I). 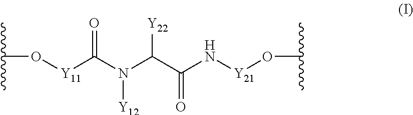
(wherein:
Y₁₁ and Y₂₁ each independently represent an alkylene group having 1 to 20 carbon(s);
Y₁₂ and Y₂₂ each independently represent a hydrogen atom or an alkyl group optionally substituted with an amino group; or
Y₁₂ and Y₂₂ are combined with each other at their terminals to represent an alkylene group having 3 to 4 carbons;
the terminal oxygen atom bound to Yᵢᵢ is bound to the phosphorus atom of the phosphate ester at the terminal nucleotide in any one region of said Y1 region and said Y2 region; and
the terminal oxygen atom bound to Y₂₁ is bound to the phosphorus atom of the phosphate ester at the terminal nucleotide in the other region of said Y1 region and said Y2 region which is not bound to Y₁₁.)

4. The method according to claim 2, wherein
said Ly linker region is a divalent group represented by the following formula (I); and
said Lz linker region is a divalent group represented by the following formula (I′). 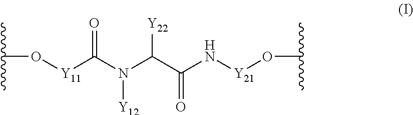
(wherein:
Y₁₁ and Y₂₁ each independently represent an alkylene group having 1 to 20 carbon(s);
Y₁₂ and Y₂₂ each independently represent a hydrogen atom or an alkyl group optionally substituted with an amino group; or
Y₁₂ and Y₂₂ are combined with each other at their terminals to represent an alkylene group having 3 to 4 carbons;
the terminal oxygen atom bound to Y₁₁ is bound to the phosphorus atom of the phosphate ester at the terminal nucleotide in any one region of said Y1 region and said Y2 region; and
the terminal oxygen atom bound to Y₂₁ is bound to the phosphorus atom of the phosphate ester at the terminal nucleotide in the other region of said Y2 region and said Y1 region which is not bound to Y₁₁.) 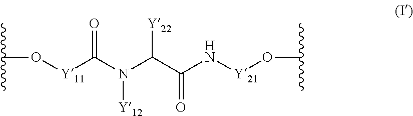
(wherein:
Y′₁₁ and Y′₂₁ each independently represent an alkylene group having 1 to 20 carbon(s);
Y′₁₂ and Y′₂₂ each independently represent a hydrogen atom or an alkyl group optionally substituted with an amino group; or
Y′₁₂ and Y′₂₂ are combined with each other at their terminals to represent an alkylene group having 3 to 4 carbons;
the terminal oxygen atom bound to Y′₁₁ is bound to the phosphorus atom of the phosphate ester at the terminal nucleotide in any one region of said Z1 region and said Z2 region; and
the terminal oxygen atom bound to Y′₂₁ is bound to the phosphorus atom of the phosphate ester at the terminal nucleotide in the other region of said Z2 region and said Z1 region which is not bound to Y′₁₁.)

5. The method according to claim 2, wherein
said Ly linker region and said Lz linker region each independently represent a divalent group having the structure represented by the following formula (II-A) or (II-B). 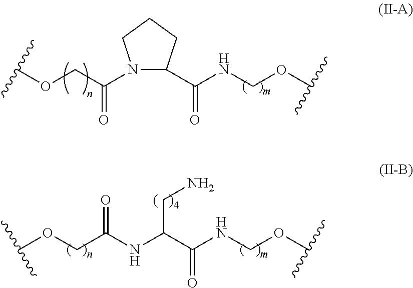
(wherein n and m each independently represent any one integer of 1 to 20.)

6. The method according to claim 1, wherein the Ly linker region is a linker region consisting of a nucleotide sequence having a smaller number of nucleotide(s) than the sum of the number of nucleotides of the X2 region, the Y2 region, the Y1 region, and the X1 region.

7. The method according to claim 1, wherein
the Ly linker region consists of a Lya region, a Lyb region, and a Lyc region from the 5′-terminal; and
the Lya region and the Lyc region are linker regions consisting of nucleotide sequences having 2 bases which do not form a Watson-Crick base pair with each other.

8. The method according to claim 1, wherein
the Ly linker region consists of a Lya region, a Lyb region, and a Lyc region from the 5′-terminal;
the Lyb region consists of a 0- to 20-mer nucleotide sequence;
the Lya region and the Lyc region each represent a linker region of a nucleotide sequence having 2 bases; and
the combination of (Lya, Lyc) or (Lyc, Lya) is selected from the following combinations.
(Lya, Lyc) or (Lyc, Lya)=(AA, AA), (AA, AC), (AA, AG), (AA, CA), (AA, CC), (AA, CG), (AA, GA), (AA, GC), (AA, GG), (AC, AA), (AC, AC), (AC, AG), (AC, CA), (AC, CC), (AC, CG), (AC, UA), (AC, UC), (AC, UG), (AG, AA), (AG, AC), (AG, AG), (AG, GA), (AG, GG), (AG, UA), (AG, UC), (AG, UU), (AU, CA), (AU, CC), (AU, CG), (AU, GA), (AU, GC), (AU, GG), (AU, UA), (AU, UC), (AU, UG), (AU, UU), (CA, AA), (CA, AC), (CA, AU), (CC, AA), (CC, AC), (CC, AU), (CC, CC), (CC, CU), (CC, UA), (CC, UC), (CC, UU), (CG, AA), (CG, AC), (CG, AU), (CG, GA), (CG, GC), (CG, GU), (GA, AA), (GA, AG), (GA, GG), (GA, GU), (GC, AA), (GC, AG), (GC, AU), (GC, CA), (GC, CG), (GC, CU), (GC, GA), (GC, GG), (GC, GU), (GC, UA), (GC, UG), (GC, UU), (GG, AA), (GG, AG), (GG, AU), (GG, GA), (GG, GG), (GG, GU), (GG, UU), (GU, CA), (GU, CG), (GU, GU), (GU, UA), (GU, UG), (GU, UU), (UA, AC), (UA, AG), (UA, AU), (UC, AC), (UC, AG), (UC, AU), (UC, CC), (UC, CG), (UC, CU), (UC, UC), (UC, UG), (UC, UU), (UG, AA), (UG, AC), (UG, AG), (UG, AU), (UG, GC), (UG, GG), (UG, GU), (UG, UC), (UG, UG), (UG, UU), (UU, CC), (UU, CG), (UU, CU), (UU, GC), (UU, GG), (UU, GU), (UU, UC), (UU, UG), and (UU, UU)

9. The method according to claim 1 wherein at least one of a W1 region consisting of said X1 region, said Y1 region, and said Z region, and a W2 region consisting of said X2 region and said Y2 region comprises a nucleotide sequence which suppresses the expression of a target gene in an RNA interference method.

10. The method according to claim 1, wherein said RNA ligase is T4 RNA ligase 2 derived from T4 bacteriophage, ligase 2 derived from KVP40, Trypanosoma brucei RNA ligase, Deinococcus radiodurans RNA ligase, or Leishmania tarentolae RNA ligase.

11. The method according to claim 1, wherein said RNA ligase is an RNA ligase consisting of an amino acid sequence having 95% or more identity to an amino acid sequence of SEQ ID NO:9, 10, or 11.

12. The method according to claim 1, wherein said RNA ligase is T4 RNA ligase 2 derived from T4 bacteriophage or RNA ligase 2 derived from KVP40.

13. The method according to claim 1, wherein said ammonium salt is at least one ammonium salt(s) selected from the group consisting of hexylammonium salts, dipropylammonium salts, dibutylammonium salts, and diamylammonium salts.

14. The method according to claim 1, wherein the filler of said reverse-phase column chromatography is a silica or a polymer on which any one or more of a phenyl group, an alkyl group having 1 to 20 carbon(s), or a cyanopropyl group is/are fixed.
